(19) United States
(12) Patent Application Publication
(10) Pub. No.: US 2013/0263298 A1
(43) Pub. Date: Oct. 3, 2013

(54) MODELS FOR DIAGNOSIS, PREVENTION AND TREATMENT OF ALZHEIMER'S DISEASE

(71) Applicant: INDIGO CAPITAL GROUP LLC, Plano, TX (US)

(72) Inventors: Lisa P. MICHAELS, Plano, TX (US); Tess P. Michaels, Plano, TX (US)

(73) Assignee: INDIGO CAPITAL GROUP LLC, Plano, TX (US)

(21) Appl. No.: 13/851,362

(22) Filed: Mar. 27, 2013

Related U.S. Application Data
(60) Provisional application No. 61/616,825, filed on Mar. 28, 2012.

(51) Int. Cl. C12Q 1/48

(52) U.S. Cl. CPC C12Q 1/48
USPC 800/12; 435/15; 424/94.4; 514/17.8; 424/752; 424/754; 424/729; 424/756; 514/690; 514/562; 514/733

(57) ABSTRACT

A transgenic fly whose genome is modified to express enhanced levels of glutamate-cysteine ligase (GCL) gene is provided. The fly displays phenotypes associated with Alzheimer's disease (AD). Further, a method for diagnosing AD is provided, which includes assessing enzymatic activities in mitochondrial enzymes. Glutathione pathway are investigated by creating Alzheimer's model Drosophila with over-expression of the GCLc gene, inducing redox stress through sleep deprivation, and analyzing mitochondrial electron transport chain (ETC) using colorimetric enzymatic assays. For prevention of AD, it is proposed that the epigenetic approaches be used to increase glutathione levels in vivo before the onset of AD. For treatment of AD, it is proposed that the glutathione levels be increased by GCLc modulation.

### CROSS-REFERENCE TO RELATED APPLICATIONS

This application claims priority of U.S. Provisional Application No. 61/616,825 filed on Mar. 28, 2012; the entire disclosure of which is incorporated herein by reference.

### BACKGROUND

1. Field of the Invention

The present invention relates generally to study, diagnosis, prevention and treatment of Alzheimer's disease (AD). More particularly, the present invention relates to an integrative model for study, diagnosis, prevention and treatment of AD that builds upon existing hypotheses on redox stress, mitochondrial dysfunction, and amyloid toxicity.

2. Description of the Related Art

Life expectancy is a good indicator of a nation's health. The life expectancy has increased dramatically around the world over the past century, mainly due to advancements in medicine and better diets. In the US, the life expectancy saw significant improvements during 1900-2000 from 47.3 to 77.3 years. However, over the past decade, the increase has been just 1.1 years, making some researchers believe that we may have peaked. The early onset of diabetes, cancers, cardiovascular and neurodegenerative diseases of aging has kept US behind 40 other nations in life expectancy. The research on aging is essential and complementary to research on diseases of aging, and can help find ways to postpone and maybe eliminate the diseases of aging, such as Alzheimer's disease (AD).

AD is the sixth leading cause of death in the US, and has grown faster than all other diseases of aging. It is the most common form of senile dementia. Till date, no known cure for this disease exists. AD worsens as it progresses, and eventually causes death of a patient. 5.4 million people in US are affected by AD, which results in an expenditure of at least $200 billion on direct care every year. By 2050, the number of AD patients in US is estimated to be 11.3 million, if no reliable method of diagnosis and cure is found. National Institutes of Health spends about $500M annually on Alzheimer's research; and in 2010, the National Alzheimer's Project was launched with a plan to overcome the disease by 2025.

### Causes:

AD is often attributed to misfolding of proteins in the brain, which results in accumulation of abnormally folded amyloid-beta and tau proteins. Misfolded amyloid-beta proteins cause plaques in the neuronal tissue, which are made up of small peptides, 39-43 amino acids in length, also known as beta-amyloid (Aβ). Aβ is a fragment of a larger protein, amyloid precursor (APP). APP is a transmembrane protein that penetrates through the neuronal cell membrane. In AD, an enzymatic process involving secretases causes cleavage of APP into smaller segments, which gives rise to beta-amyloid, which forms dense senile plaques, causing AD.

### The Problem

In spite of continued efforts by many researchers, the development of effective ways to diagnose, treat or prevent AD remains elusive. Current biomarkers are mostly behavioral, and the therapeutic strategies are limited to those that attenuate AD symptomology without deterring the progress of the disease itself, and thus only postpone the inevitable deterioration of the affected individual.

Currently, the diagnosis of AD as outlined in the National Institute of Neurological and Communicative Disorders and Stroke (NINCDS) and the Alzheimer's Disease and Related Disorders Association (ADRDA) criteria is based on clinical neuropsychological examinations, identification of common symptoms of AD and exclusion of known causes of AD. However, these criteria fail to consider latest developments in diagnosis and treatment of neurodegenerative disorders. Moreover, they underestimate the value of biomarkers in diagnosis of AD.

Further, due to variable nature of clinical phenotypes, accurate diagnosis of AD is not always possible, especially in its early stages. One of the underlying reasons may be the lack of brief screening tests adequately validated for early stage detection of AD. Therefore, there exists a need to provide measurable in vivo biomarkers for early stage detection and prognosis of dementia.

A number of biomarkers, such as MRI, PET scans and cerebrospinal fluid (CSF) have been proposed for diagnosis of likelihood that AD is present. Such biomarkers are often invasive and uncomfortable, measuring them entails high costs, and thus, their use may not be readily available to all.

Criteria for an ideal biomarker of AD have been proposed by a consensus group on molecular and biochemical markers of AD; “The ideal biomarker for AD should detect a fundamental feature of neuropathology and be validated in neuropathologically confirmed cases; it should have a diagnostic sensitivity of more than 80% for detecting AD and a specificity of more than 80% for distinguishing other dementias; it should be reliable, reproducible, noninvasive, simple to perform, and inexpensive.”

To date, proposed therapeutic strategies have failed to demonstrate long-term efficacies. Therapies oriented towards anti-Aβ or anti-oxidant strategies have not been very successful. However, few studies report that individuals with mid-to-late stage AD have been administered anti-Aβ immunotherapy that cleared a large proportion of brain amyloid plaques; however, this evidence did not seem to alter the presence of neurofibrillary tangles (NFTs) or the inexorable course of the disease. Neocortical NFTs and cognitive impairment exhibit the best correlation; however, no therapies that target cortical NFTs have been successfully tested in humans. As hypothesized, the current clinical trials indicate that there exists a patho-genetic synergy in which amyloid plaques kindle an auto-propagating process related to cortical NFTs. For now, clinical trials have not provided definitive answers either way.

Several exogenous antioxidants, when included in diet, lower the risk for AD. While this may suggest that people at the risk for developing AD or being in the early phases of AD may benefit from intervention of exogenous antioxidants, current clinical studies do not provide a definitive answer to whether antioxidants are truly protective against AD.

A number of in vitro and in vivo studies in laboratory animals propose natural antioxidants, such as resveratrol, curcumin and acetyl-l-carnitine as alternative therapeutic agents for AD. Many other studies demonstrated the efficacy of primary antioxidants, such as polyphenols, or secondary antioxidants, such as acetylcarnitine, in reduction and blocking of neuronal death caused due to AD. These studies revealed that mechanisms other than the antioxidant activities could be involved in the neuroprotective effect of these compounds. New drug delivery systems and association thereof may be useful for the therapeutic use of antioxidants in AD.

### New Approaches:

Mitochondria have been an area of focus for many researchers for exploring mitochondrial dysfunctions related to a number of diseases, as these organelles drive many cellular processes, such as energy metabolism, apoptosis, and aging. Human cells contain up to a thousand mitochondria. Studies have shown that mitochondria are sites of damage in AD. Mitochondria are early targets of Aβ aggregates, and elevated Aβ toxicity impairs the electron transport chain (ETC).

ETC related defects that may be associated with antioxidant imbalance are thought to cause energy metabolism related defects and induce cellular degeneration. Mitochondrial proteins act as binding sites for Aβ, resulting in a toxic response. Cells may be protected at the mitochondrial level, by stabilizing or restoring mitochondrial function and energy homeostasis.

Further, free radicals generated through mitochondrial metabolism may cause abnormal function and cell death. Mitochondria are an important source of oxidants for a cell. Approximately 1-2% of electron flow through the ETC results in monovalent generation of superoxide. Moreover, various toxins in the environment injure mitochondrial enzymes, leading to increased generation of free radicals that play a major role in aging.

The aging process may weaken the mitochondrial oxidative system, providing a basis for the specific and destructive effects of Aβ and tau. In mitochondrial respiration, synthesis of adenosine triphosphate (ATP) necessary for endergonic reactions, and generation of reactive oxygen species are two contradictory functions performed by mitochondria for cell survival. The latter may severely affect long-term survival of cells and constitutes a major underlying cause of the aging process. During aging, free radicals escape, as some of the free radical-scavenging systems are decreased. This decreases the fluidity and increases the permeability of the inner mitochondrial membrane.

Oxidatively damaged proteins including functionally inactive forms of enzyme are known to increase with age, as result of free radical-induced damage and consequent DNA damage. This in turn affects the factors responsible for protein oxidation and subsequent degradation thereof. Accumulation of oxidized proteins may result in cross-linking with other proteins that would alter biochemical and physiological mitochondrial functions. Aggregated Aβ-protein in diseased brains may get accumulated as neurofibrillary tangles can occur by way of oxidative reactions. Further, the content of protein carbonyls in AD brain samples is greater than that in age-matched controls, providing a clear indication of greater accumulation of oxidized proteins in AD.

Mitochondrial ETC forms a potential basis for AD pathogenesis. In the ETC, complexes I and III leak electrons to oxygen, producing superoxide radicals which in turn produce H₂O₂. In addition, ETC involves H₂O₂ reducing to H₂O and O₂ by glutathione peroxidase, which yields energy to generate ATP from ADP and inorganic phosphate. Impaired complex IV activity has also been demonstrated in AD. Increased free radical-induced oxidative stress has been associated with the development of AD, in which nitrogen monoxide may play a critical role. Nigral GSH loss is considered an early and crucial event in the pathogenesis of Parkinson's disease, resulting in peroxynitrite scavenging.

The redox pathway in mitochondria involves all the five enzyme complexes. Data from studies of rat brain mitochondria have shown that complex activities need to be reduced by at least 60% before major changes in ATP synthesis and oxygen consumption occur. Complex I, III and IV activities have to be decreased 25%, 80% and 70%, respectively, before rates of oxygen consumption and ATP synthesis change significantly. These results suggest that in mitochondria of synaptic origin, complex I activity has a major control of oxidative phoshorylation, such that when a threshold of 25% inhibition is exceeded, energy metabolism is compromised, and reduction in ATP synthesis occurs. Moreover, the same study demonstrated that depletion of glutathione abolished the threshold for complex I, providing experimental evidence that antioxidant status is critically involved in maintaining energy thresholds in mitochondria.

Furthermore, inherited or acquired mutations impair ETC functioning, leading to decreased ATP production, and increased formation of free radicals. This further sustains mitochondrial damage, including oxidation of mitochondrial DNA, proteins, and lipids, resulting in cell degeneration and death. Oxidative stress alters the expression of antioxidant enzymes. Moreover, it is well known that brain cells are continually challenged by conditions which may cause acute or chronic stress.

Aβ accumulation is viewed as a downstream event and addressing Aβ accumulation by blocking production, enhancing removal, or preventing oligomerization have limited effects. Such interventions help to some degree if Aβ forms a part of a feedback loop that reduces mitochondrial respiration in already failing mitochondria. Though, eliminating Aβ would not correct the primary problem, it would not be expected to dramatically alter clinical progression.

Accumulating evidence from both animal and human studies indicates a major role for oxidative damage in the pathogenesis of AD, occurring even before symptoms arise and both beta-amyloid-containing plaques and neurofibrillary tangles are formed. This raises the possibility of preventing or at least slowing down the progression of AD by the use of antioxidants.

The Central Nervous System (CNS) has a large potential oxidative capacity due to the high level of tissue oxygen consumption. However, the ability of the brain to withstand oxidative stress is limited because of relatively low levels of antioxidants such as glutathione and antioxidant enzymes (such as glutathione peroxidase, catalase and superoxide dismutase) and endogenous generation of reactive oxygen free radicals via several specific reactions. CNS contains non-replicating neuronal cells. If these neuronal cells are damaged, they may become permanently dysfunctional or lead to programmed cell death (apoptosis).

### Role of GCLc—GSH

GCL, also known as g-glutamylcysteine synthetase (GCS), is the first rate limiting enzyme of glutathione synthesis. GCLc is the catalytic subunit that is an enzyme that in humans is encoded by the GCLc gene and maps to chromosome 6. It is up-regulated by many cellular stresses. Some known inducers of GCL include nitric oxide, and quinones.

Glutathione (C₁₀H₁₇N₃O₆S) is synthesized de novo by the consecutive action of two enzymes, viz., glutamatecysteine ligase-catalytic subunit (GCLc) (gamma-glutamyl cysteine synthetase), which catalyzes the first and rate-limiting step in the synthesis of glutathione and by GSH synthase. The determinants of Glutathione synthesis are the availability of cysteine, activity of GCLc enzyme, and feedback mechanisms.

L-Glutamate+L-cysteine+ATP←GCLc enzyme→L-gamma-glutamyl-Lcysteine+ADP+Pi Reaction 1:

L-gamma-glutamyl-Lcysteine+Glycine+ATP←GSH synthase→Glutathione+ADP+Pi Reaction 2:

Induction of GCLc gene primarily controls sustained increases in GSH content. Many studies suggest that redox-depending signalling pathways control the expression of GCLc. Further, GCL enzymes detoxify reactive oxygen species (ROS) via activation of glutathione peroxidase and elevation of thiols. Decreased expression of GCLc is associated with AD.

The tripeptide glutathione (GSH) is the most important intracellular antioxidant that is present in highest concentration in cells of all organs. The content of GSH within the cell varies with stress. In many cases, adaptation to subsequent stressors is caused due to a process involving GSH synthesis. GSH is an endogenous antioxidant of utmost importance that is required for the maintenance of the thiol redox status of the cell, protection against oxidative damage, detoxification of endogenous and exogenous reactive metals and electrophiles, storage and transport of cysteine, as well as for protein and DNA synthesis, cell cycle regulation and cell differentiation. Glutathione and glutathione-related enzymes play a key role in protecting the cell against the effects of ROS. Glutathione has many important roles in cell function, including regulating redox-dependent signal transduction pathways. The key functional element of glutathione is the cysteinyl moiety, which provides the reactive thiol group. Glutathione is the predominant defence against ROS, which are reduced by GSH in the presence of GSH peroxidase. As a result, GSH is oxidized to Glutathione disulfide (GSSG), which in turn is rapidly reduced back to GSH by GSSG reductase at the expense of nicotinamide adenine dinucleotide phosphate (NADPH). The thiol-disulfide redox cycle also aids in maintaining reduced protein and enzyme thiols.

Glutathione also aids in the storage and transfer of cysteine. Cysteine auto-oxidizes rapidly to produce toxic oxygen radicals. To avoid the toxicity of cysteine, most of the non-protein cysteine is stored in glutathione. Glutathione is also a good scavenger of lipid peroxidation products that bind proteins inhibiting their activities. Glutathione also reacts with saturated carbon atoms (epoxides), unsaturated carbon atoms (quinones, esters), and aromatic carbon atoms (aryl nitro compounds). This detoxification involves nucleophilic attack by GSH on an electrophilic carbon. This reaction is often catalysed by glutathione S-transferase. GSH functions in storage, mobilization and delivery of metal ions between ligands, in the transport of metal across cell membranes, as a source of cysteine for metal binding, and as a reductant in redox reactions involving metals. Glutathione reacts with free radicals; therefore, increased glutathione levels may prove beneficial against oxidative stress.

There are evidences of impairment in vivo of glutathione homeostasis and antioxidant enzymes in patients with AD, suggesting a relevant role of free radical cytotoxicity in the pathophysiology of the disease.

Thus, the “triple cascade” of amyloid toxicity, redox stress, and mitochondrial dysfunction triggers amyloid plaques and tau tangles through a number of pathways, such as elevation of reactive oxygen species production, and interaction with mitochondrial proteins, contributing to the development and progression of AD.

A newer field of aging research is known as epigenetics. Reversible changes have been reported in human genes that can modify gene expression. Epigenetics is the study of changes in gene activity that do not involve alterations to the genetic code but still get passed down to at least one successive generation. These patterns of gene expression are governed by the cellular material, the epigenome, which consists of a record of chemical changes that can be passed down to an organism's offspring. It is these epigenetic ‘marks’ that regulate an organism's gene expression. Environmental factors, such as diet, stress, and prenatal nutrition can make an imprint on genes that is passed from one generation to the next.

### Animal Models

One of the most important challenges is to find more appropriate animal model systems in which genetic or epigenetic manipulation is possible and to identify clear characteristics that will allow the aging phenotype to be measured.

There are many animal models of AD including flies, mice, rats, rabbits, canines, and non-human primates. Each model seems to mimic different aspects of AD. Most transgenic mice that overproduce Aβ successfully mimic extracellular amyloid deposits, behavioral deficits and memory defects, but they do not exhibit global neuronal loss.

The dominating role of the mouse in modeling Alzheimer's disease has been challenged by Drosophila melanogaster. The fruit fly Drosophila has been widely used as a model organism to investigate many phenomena related to biology and biochemistry. Drosophila genome-based studies suggest that approximately 70% of human disease related genes have homologs in Drosophila.

Many neurological and neurodegenerative disorders in humans can be studied based on Drosophila genetic models. For AD-like diseases caused by misfolding of proteins, an overexpression of some genes in Drosophila neurons has been shown to mimic key aspects of such diseases. Drosophila includes a homolog of APP, known as APP-like protein (dAPPL). APP is cleaved to release Aβ domain by α-secretase precluding deposition of intact Aβ peptide. Kuzbanian is the Drosophila ortholog of α-secretase ADAM10 that cleaves dAPPL. Drosophila β-secretase-like enzyme dBACE has similar identity to human BACE1 and BACE2 that cleave human APP. An overexpression of dBACE in Drosophila cleaves the dAPPL and produces a fragment corresponding to Aβ peptide. Human Aβ42 peptides can be overproduced in fly neurons by a) overexpression of Aβ using genetic methods or b) directly injecting Aβ protein into tissue. Direct protein injection methods have shown to result in stable transmission of deleterious effects of the accumulated Aβ through several generations. Using a number of assays, an age-dependent memory defect (based on assessment of sensory systems) in Aβ42 flies can be observed. This has been directly correlated with human AD, and therefore, an Aβ42 Drosophila model can be used in the diagnosis of and therapeutic avenues for AD.

GSH can be synthesized de novo by the consecutive action of two enzymes, glutamate-cysteine ligase (GCL) and GSH-synthase (GS). Mammalian GCL is a heterodimeric enzyme, consisting of a catalytic subunit, GCLc and a regulatory or modulatory subunit, GCLm. GCLc is encoded by the GCLC gene. GSH provides a protection against oxidative stress in vivo. A Drosophila model can be developed using global and tissue-specific promoters, to achieve overexpression of Drosophila GCL catalytic and modulatory subunits. A GCLc overexpressed fly model shows an enhancement in the ability of Drosophila to synthesize GSH and results in 50% prolonged life-span of Drosophila.

An increase in Aβ42 levels above a given threshold in the brain is generally regarded to be the primary event in AD pathogenesis, and approaches to develop disease-modifying therapies have focused on lowering Aβ42 levels. The steady state level of Aβ42 reflects the balance between production and clearance of Aβ42, and an imbalance of these activities could be sufficient to raise Aβ42 levels. Thus, reducing Aβ42 levels can be achieved either by attenuating production, or by facilitating degradation and/or clearance of Aβ42 from the brain. The Aβ42-GCLc fly model thus provides a unique tool for studying the mechanism underlying intraneuronal Aβ42 accumulation and neurodegeneration.

### Description of the Related Patents

US patent application serial number 2006/0228728, titled “Genetic basis of Alzheimer's disease and diagnosis and treatment thereof”, by Cox, et. al., describes methods of polymorphic profiling in various haplotype blocks that have at least two genes. The total number of resistance and susceptibility alleles in the polymorphic profile is determined, which provides an indication of whether an individual has or is at risk of Alzheimer's disease. The method uses the genes and encoded proteins to identify compounds that modulate the expression or activity of encoded proteins. Such compounds are used in treatment, prophylaxis, diagnosis or prognosis of Alzheimer's related diseases.

European patent serial number EP2557421A2, titled “Altered mitochondrial activity in diseases, resulting from oxidative stress”, and serial number EP2142923A2, titled “Altered mitochondrial activity in diabetic nephropathy”, by Malik describe a method for identifying a subject having or at risk of developing Alzheimer's disease resulting from oxidative stress. At least one indicator of altered mitochondrial activity is measured in a test sample. A difference in mitochondrial activity in the test sample as compared to a control is indicative that said subject has or is at risk of developing Alzheimer's disease. The inventions use cytochrome oxidase (complex IV) and complexes I, II, and III for conducting enzymatic assays. However, Malik does not address any aspect related to prevention and/or treatment of the Alzheimer's disease.

European patent serial number EP1774972B1, titled “Use of selenium yeasts in the treatment of Alzheimer's disease”, by Pearse, et. al., describes the use of a composition comprising Sel-Plex in the manufacture of a medicament for a treatment for Alzheimer's disease. The medicament is formulated for administration to a subject under conditions, such that the expression of a complement gene is reduced in the cerebral cortex of said subject. Selenium can be used as a trace element involved in regulating aspects of the antioxidant defence mechanism in all living tissues by interacting with the body's glutathione (GSH) and its major Se-containing antioxidant enzymes, glutathione peroxidase (GPX) and thioredoxin reductase. The complement gene is one of a cathepsin gene, a presenilin gene, and nicastrin. However, Pearse does not address any aspect related to diagnosis of Alzheimer's disease by using the inventive method.

In light of the foregoing discussion, it would be advantageous to provide an integrated model for diagnosis, prevention, and treatment of AD that builds upon existing hypotheses on redox stress, mitochondrial dysfunction, and amyloid toxicity.

### SUMMARY

An object of the present invention is to evaluate the epigenetic effects of antioxidants on the lifespan of Drosophila melanogaster, and study the genetic and biochemical pathways of GCLc and Glutathione.

Another object of the present invention is to provide a method for diagnosis, prevention and treatment of neurodegenerative disorders, such as Alzheimer's disease.

Another object of the present invention is to provide a method for diagnosis, prevention and treatment of Alzheimer's disease, which is less-invasive as compared to the current and proposed methods.

Yet another object of the present invention is to provide a method for diagnosis, prevention and treatment of Alzheimer's disease, which is less expensive as compared to the current and proposed methods.

Yet another object of the present invention is to create a transgenic model of Aβ42-GCLc Drosophila by crossbreeding beta amyloid (Aβ42) with GCLc-Gal4 Drosophila.

Still another object of the present invention is to investigate the glutathione pathway by creating Alzheimer's model Aβ42 Drosophila with over-expression of the GCLc gene, inducing redox stress through sleep deprivation, and analyzing mitochondrial electron transport chain using colorimetric enzymatic assays.

Embodiments of the present invention provide a living organism that expresses enhanced levels of an enzyme associated with antioxidant synthesis, wherein the living organism displays at least one phenotype associated with a neurodegenerative disorder, such as Alzheimer's disease. The living organism is a transgenic fly of Drosophila genus and the antioxidant used is glutathione (GSH). Further, the enzyme is glutamate-cysteine ligase (GCL). In an aspect, the enhanced level of enzyme is induced by using at least one dietary supplement, including Ginkgo biloba, Resveratrol, and Glutathione.

Embodiments of the present invention provide a method for diagnosing a neurodegenerative disorder, such as AD, in a first living organism. An enzymatic activity of at least one mitochondrial enzyme in a first sample isolated from the first living organism is assessed. The enzymatic activity is compared with an enzymatic activity of the at least one mitochondrial enzyme in a second sample isolated from a second living organism known to be free of the neurodegenerative disorder. A difference between the first enzymatic activity and the second enzymatic activity indicates a presence of the neurodegenerative disorder in the first living organism.

Embodiments of the present invention provide method for preventing and treating a neurodegenerative disorder, such as Alzheimer's disease in a living organism by increasing a level of glutathione in the living organism. The level of glutathione in the living organism is increased by modulating an expression of glutamate-cysteine ligase (GCL).

### BRIEF DESCRIPTION OF THE DRAWINGS

The following detailed description of the preferred embodiments of the present invention will be better understood when read in conjunction with the appended drawings. The present invention is illustrated by way of example, and not limited by the accompanying figures, in which like references indicate similar elements.

FIG. 1 shows a table that shows properties of various dietary supplements used in the tests performed in accordance with various embodiments of the present invention;

FIG. 2 is a graph illustrating the lifespan in days for yellow white Drosophila, fed with various dietary supplements, in accordance with an exemplary test;

FIG. 3 is a graph illustrating the lifespan in days for yellow white Drosophila, fed with selected dietary supplements provided in varying concentrations that are multiples of normal concentration, in accordance with an exemplary test;

FIG. 4 is a graph illustrating the results of GCLc enzymatic assays performed by using Western Blot Tests for the selected dietary supplements, in accordance with an exemplary test;

FIG. 5 is a graph illustrating the results of glutathione assays performed by using a fluorimeter for the selected dietary supplements, in accordance with an exemplary test;

FIG. 6 is a graph illustrating average life-span in days for yellow-white, Aβ42, and Aβ42-GCLc Drosophila, in accordance with an exemplary test;

FIG. 7 is a graph illustrating average life-span in days for stressed yellow-white, Aβ42, and Aβ42-GCLc Drosophila, in accordance with an exemplary test;

FIG. 8 is a graph illustrating the activity of complexes I and III for yellow-white, Aβ42, and Aβ42-GCLc Drosophila, in accordance with an exemplary ETC assay;

FIG. 9 is a graph illustrating the activity of complex IV for yellow-white, Aβ42, and Aβ42-GCLc Drosophila, in accordance with an exemplary ETC assay;

FIG. 10 is a graph illustrating the activity of complex II for yellow-white, Aβ42, and Aβ42-GCLc Drosophila, in accordance with an exemplary ETC assay; and

FIG. 11 is a graph illustrating the activity of complexes II and III for yellow-white, Aβ42, and Aβ42-GCLc Drosophila, in accordance with an exemplary ETC assay.

### DETAILED DESCRIPTION OF THE INVENTION

The detailed description of the appended drawings is intended as a description of the currently preferred embodiments of the present invention, and is not intended to represent the only form in which the present invention may be practiced. It is to be understood that the same or equivalent functions may be accomplished by different embodiments that are intended to be encompassed within the spirit and scope of the present invention.

The present invention, in one embodiment, aims at evaluating the epigenetic effects of antioxidants on the lifespan of Drosophila melanogaster, and studying in detail, the genetic and biochemical pathways of GCLc and Glutathione.

The previous research done on longevity and aging was reviewed, as outlined in the background section of the present patent application. The purpose, hypothesis and the research plan for this comparative longevity study was devised and a detailed scientific experiment was drawn up to complement the previous research done.

A ‘Fly Lab’ was created, complete with a temperature controlled insect culture incubator, multiple plastic K-resin vials and glass culture bottles, carbon dioxide anesthetizer, stereomicroscope, Carolina blue 424 culture media, yeast, water, gempro weighing machine, magnifying glass, fly pad, measuring cups, cotton balls, labels, markers, calculator etc. Multiple vials of Drosophila melanogaster were transferred from the SMU lab as the parent population.

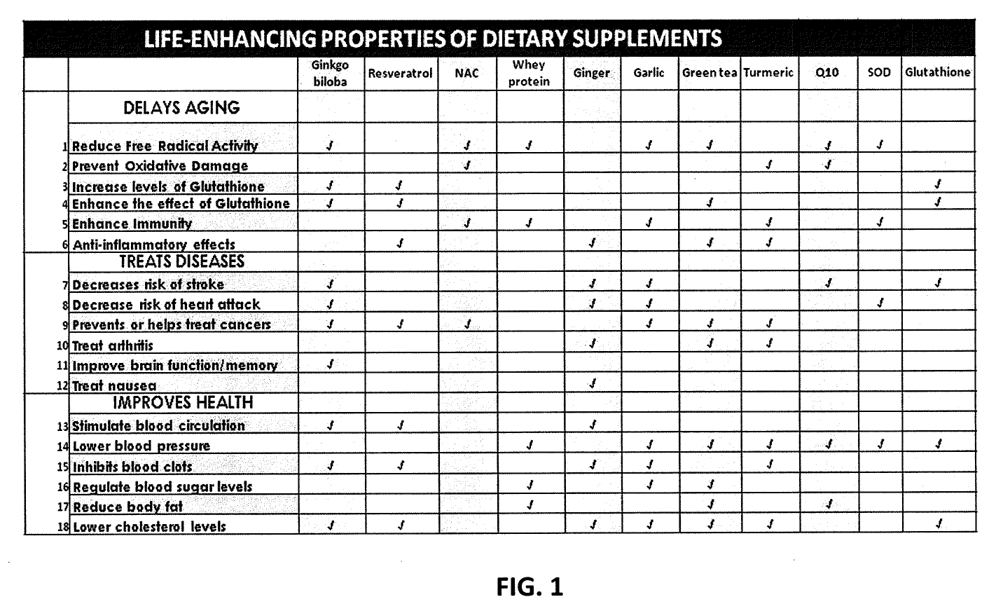

In an embodiment of the present invention, eleven dietary supplements, viz., Ginkgo biloba, Resveratrol, Glutathione, Superoxide Dismutase (SOD), and Coenzyme Q10, NAC (N acetyl cysteine), Whey Protein, Ginger, Garlic, Turmeric, and Green Tea, were used for the detailed study. FIG. 1 shows a table that shows life enhancing properties of these dietary supplements. These supplements were purchased in the purest forms available and all the required materials were gathered.

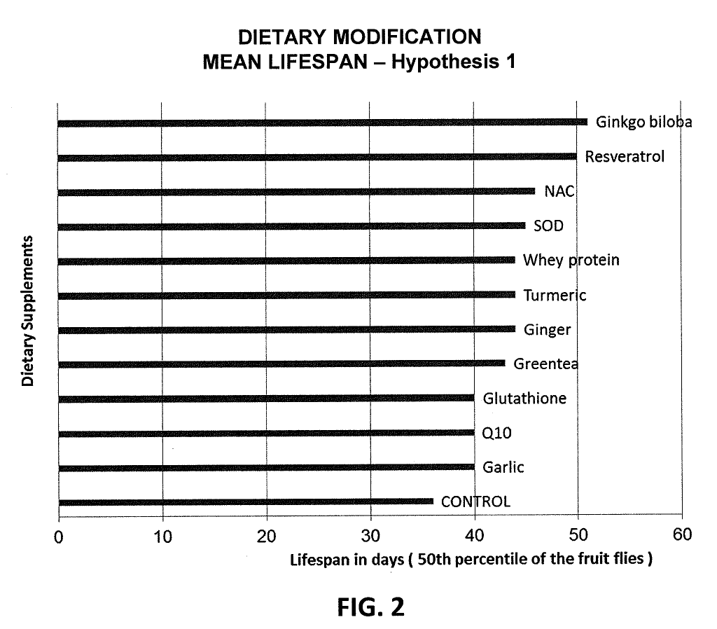

For the phase-I of the study, approximately 1200 male yellow-white Drosophila were bred, isolated and monitored on a daily basis. The life spans of these Drosophila were recorded, as shown in FIG. 2. About 100 fruit flies were tested for each supplement, divided into four vials containing 25 flies each.

In an embodiment of the present invention, a mathematical formula was developed by the inventor for an optimal dosage: Recommended daily human dosage of each supplement per day, divided by 1,000,000 (average weight of a person 50 kg, average weight of 100 flies 0.05 mg), multiplied by 2 as tubes are used initially for two days, multiplied by 100 as it is assumed only 1% of the food is consumed, multiplied by 4 as bulk food was made for 1 week/8 days. According to the formula, if 100 mg is the recommended human daily dosage of a supplement, the fly dose is 0.08 mg. This was considered optimal required daily allowance (RDA). The supplements were measured on the milligram gemological weighing machine and combined with the regular Drosophila diet (Carolina 424 media, yeast, water) in a temperature controlled insect culture incubator under optimal conditions. 50% survival was calculated by counting the flies that died and transferring the live flies into a different vial with newly made media every two days and near the end, every day. The observations were recorded, calculated, graphed and analysed. Two tailed t-tests were performed to test for statistical significance.

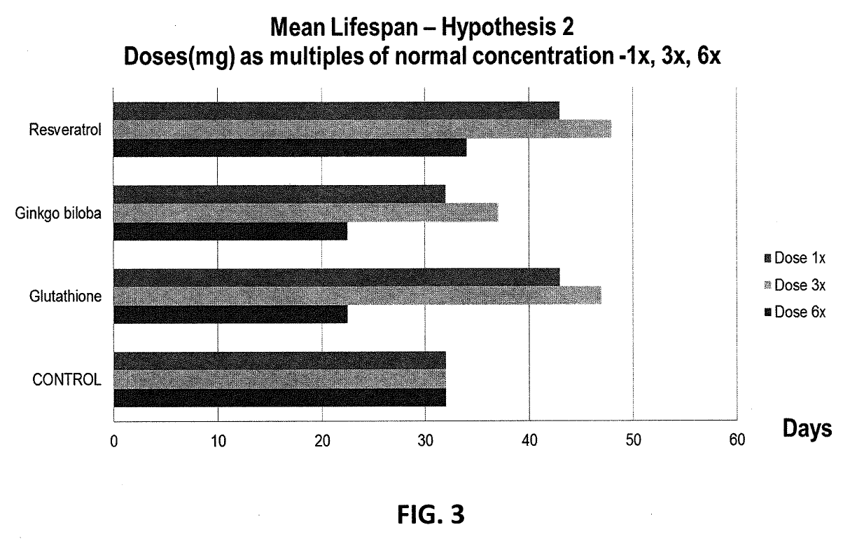

For the phase-II of the study, based on the results of the phase-I of the study, the most effective supplements for increasing longevity (Ginkgo biloba and Resveratrol) and Glutathione (used for comparison as Glutathione as a supplement does not get absorbed very well into the body and therefore, alternative supplementation is needed) were chosen for testing. In order to test the efficacy of different concentrations and to quantify the optimal dosage, single, triple, and six times the RDA for each group was tested. 50% survival was calculated, and analysed. FIG. 3 is a graph illustrating the lifespan in days for yellow white Drosophila, fed with Ginkgo biloba, Resveratrol, and Glutathione.

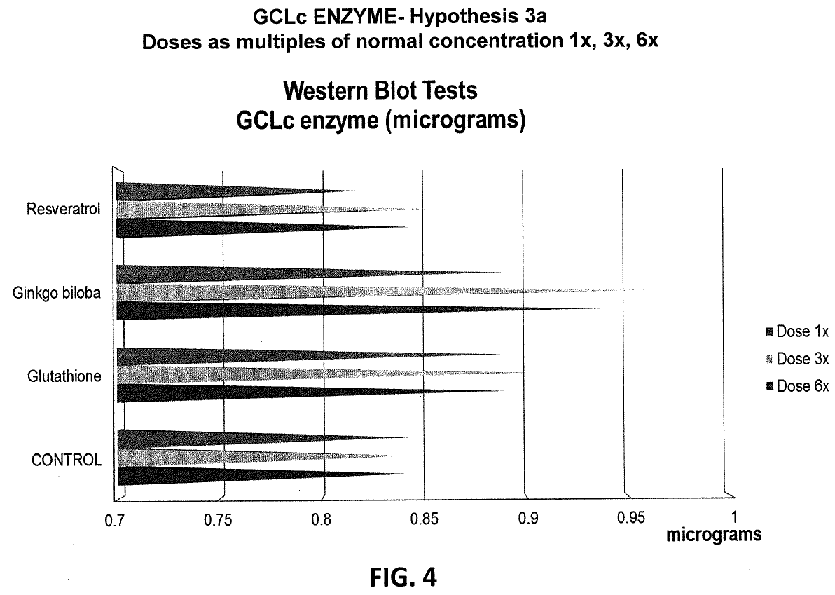

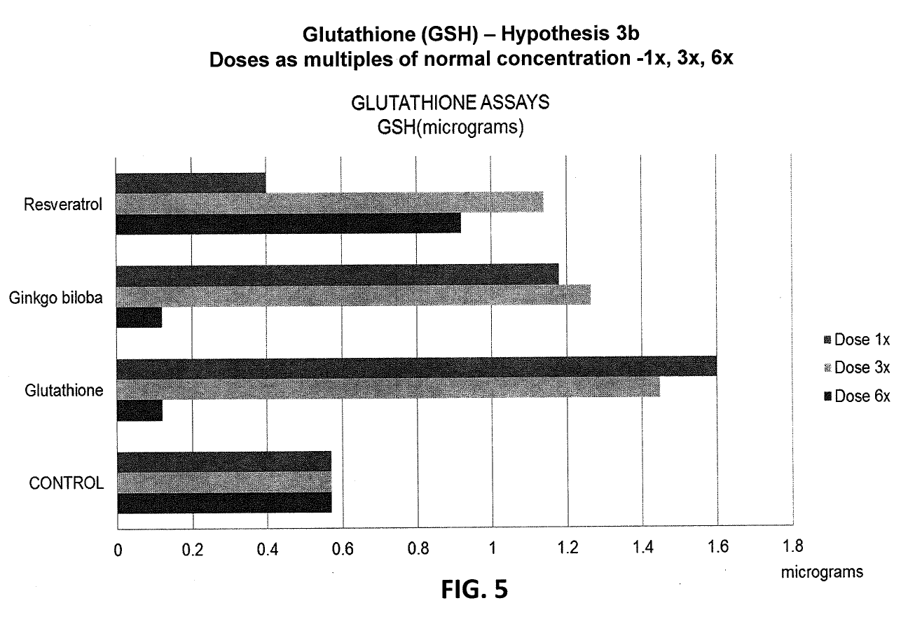

For the phase-III of the experiment, a new set of Drosophila melanogaster were bred for Western blot and Glutathione assays. There were three different concentrations used for each of the Ginkgo biloba, Resveratrol, and Glutathione groups. Single, triple, and six times strength for each of the three supplements were added to 25 flies each in vials along with control for 15 days. The flies were then transferred to perform the Western Blot test to check for GCLc enzyme levels for each group. After the Western blot test was completed, the results were quantified and graphed, as shown in FIG. 4. A Fluorometric Glutathione Assay was performed to check the Glutathione levels in each group and the results were quantified and analysed, as shown in FIG. 5.

In various aspects of the present invention, the following hypotheses were proposed and tested using scientific methods:

### Hypothesis 1: Antioxidants & Lifespan

If dietary supplements such as Ginkgo biloba, Resveratrol, Glutathione, Superoxide dismutase, Coenzyme Q10, N-Acetyl Cysteine, Whey Protein, Ginger, Garlic, Green Tea and Turmeric, are added to the diet of Drosophila melanogaster, these groups will have longer life spans as compared to the control group (yellow-white Drosophila melanogaster fed with normal diet).

### Hypothesis 2: GCLc (Genetic Pathway)

If select antioxidants Ginkgo biloba, Resveratrol, and Glutathione are added in single, triple and six times doses to the diet of Drosophila melanogaster, it will affect their life span causing dose-dependent beneficial and toxic effects as compared to the control group.

### Hypothesis 3: Glutathione (Oxidative Pathway)

3a: If select antioxidants Ginkgo biloba, Resveratrol, and Glutathione are added in optimal doses to the diet of Drosophila melanogaster, these groups will exhibit over-expression of the GCLc gene as compared to the control group, as measured by Western Blot Tests and this will be dose dependent.

3b: If the antioxidants Ginkgo biloba, Resveratrol, and Glutathione are added in optimal dose to the diet of Drosophila melanogaster, these groups will exhibit increased levels of Glutathione as compared to the control group, which is dose dependent. This will be quantified by the glutathione Fluorometric assay.

### Western Blot Tests: Hypothesis 3a

For the second hypothesis, three different concentrations were used for each of the Ginkgo biloba, Resveratrol, and Glutathione groups. Single, triple, and six times strength for each of the three supplements were added to a new batch of 25 flies each in vials along with control for 15 days.

The flies were then transferred to perform the Western Blot Test to check for GCLc enzyme levels for each group. Details of the procedure for Western Blot are mentioned below. A western blot is a method that allows investigators to detect a specific protein in a given sample of tissue.

### Tissue Preparation

The flies were frozen with dry ice, then standardized, lysis buffer added, homogenized and then centrifuged.

### Gel Electrophoresis

The proteins were separated by their molecular weight (GCLc-70KD) by SDS-PAGE, polyacrylamide gel electrophoresis. An 8% acryl amide gel was used. Smaller proteins migrate faster. Samples were loaded into the wells and a voltage of 150 Volts was applied.

### Transfer

The proteins were transferred to a PVDF sheet of special blotting paper. The uniformity was checked by the Coomassie stain.

### Blotting

The blot was incubated with a generic protein (milk proteins) to bind to any remaining sticky places.

### Detection

The primary antibody was incubated to the solution overnight, washed and the secondary targeting antibody added. This antibody had an enzyme or dye attached to it. A digital image of the western blot was captured using fluorescence and analysed.

### Glutathione Assay: Hypothesis 3b

### Reagent Preparations:

Glutathione Detection Kit from BioVision's ApoGSH was used for an in vitro assay for detection of total glutathione changes in tissue samples. The assay used monochlorobimane (MCB), a dye that appears to form an adduct exclusively with glutathione. The unbound MCB is almost non-fluorescent, whereas the dye fluoresces is blue when bound to glutathione. The reaction was catalysed by glutathione S-transferase. Thus, the amount of total glutathione was detected using a fluorimeter.

In an embodiments of the present invention, the kit contents included Cell Lysis Buffer—25 ml, Monochlorobimane (25 mM)—200 μl, GST Reagent (50 U/ml)-200 μl, and GSH Standard (1 mg; MW: 307)—1 Vial.

Monochlorobimane was dissolved in DMSO and warmed at a temperature above 18° C. for use. After opening the kit, MCB, GST, and GSH were stored at −20° C. and Cell Lysis Buffer was stored at 4° C. The GSH standard was reconstituted with 100 μl dH₂O to generate 10 μg/μl standard stock solution. It was frozen immediately after each use.

### Sample Preparation:

In an embodiment of the present invention, the cells or tissues included Lyse 1×106 cells or 10 mg tissues in 100 μl Cell Lysis Buffer.

### Assay Protocol:

Standard Curve Preparation: 10 μl of the reconstituted 10 μg/μl standard GSH stock solution was diluted into 990 μl Cell Lysis Buffer to generate 0.1 μg/μl Standard GSH solution (fresh dilute was used each time). 0, 2, 4, 6, 8, and 10 μl was added into each individual wells of a 96-well plate to generate 0; 0.2; 0.4; 0.6; 0.8; 1.0 μg/well glutathione standard. Cell Lysis Buffer was added to a volume of 100 μl for each well.

Samples: assay samples were diluted with Cell Lysis Buffer to a total volume of 100 μl in the 96-well plate. Several dilutions were included for each sample.

Reaction: 2 μl of the 50 U/ml GST Reagent and 2 μl of MCB dye were added into each sample and standard separately, and mixed well. The reaction was incubated at 37° C. for 30 min.

Reading: The fluorescence value was measured in a fluorimeter at Ex./Em.=380/460 nm. The sample readings were applied to the standard curve to calculate total glutathione amount in each sample. The results were expressed in ng/ml of sample; or ng/106 cells.

The data and results strongly support the hypotheses and offer significant evidence that the epigenetic effects of optimal dietary modification can increase longevity and may act through the GCLc/Glutathione pathway.

### Dietary Supplements—Hypothesis 1: Strongly Supported

Hypothesis 1: The data and the results shown in FIG. 2 strongly supported the hypothesis. Ginkgo biloba and Resveratrol were very significant, Ginkgo biloba increasing mean survival by 42% (51 days vs. 36 days control) and Resveratrol by 39% (50 days vs. 36 days control). NAC, SOD, Whey protein, Turmeric, Ginger, Green Tea, had respectively 28%, 25%, 22%, 22%, 22% and 19% increase in longevity, which were all statistically significant at the 99% level. Glutathione, Coenzyme Q10 and Garlic increased longevity by 11% which was not statistically significant.

Hypothesis 1 Statistical Analysis: The life spans of the 11 supplements were compared with the control group with unpaired student's t-tests using Microsoft Excel which reports the ‘p-value’. The Excel program reported p values<0.01 corresponding to a confidence Level of 99% for all supplements except for Garlic, Glutathione, and Coenzyme Q10.

Hypothesis 2: As can be seen from FIG. 3, increasing the concentration to triple and six times for Ginkgo biloba, Resveratrol, and Glutathione showed interesting results compared to the control life span of 32 days. Three times dosage had the most positive effect on longevity. 6 times dosage had inverse effects on the lifespan for all the supplements, and toxic effects for all, except Resveratrol. The life span of Ginkgo biloba was highest at the three times dose, 47 days, but six times dosage dramatically reduced the life span to 27 days. These results confirm the importance of supplementing with optimal dosages.

Hypothesis 2 Statistical Tests: The Excel program reported the ‘p-value’ as well as the t-statistic. Two-tailed t-tests were run for each of the supplements vis-à-vis the data for the control group. The p values were far below the risk level of 1% (99% confidence level) set for this project, for all supplements except Glutathione which supports the theory that natural glutathione is not easily absorbed in the body, as shown in Table. 1. The survival rates of the three groups are independently compared with the control group. The two-tailed t-tests evaluate if the means of the two groups are statistically different from each other. The t-statistic is the ratio of the ‘difference between means’ divided by the ‘variability of the groups’. The test assumed that the two groups of the data have similar distribution, which holds true for this data sheet.

TABLE 1
Statistical support for hypothesis 2
Statistical Support - Hypothesis 2
Doses as multiple of normal concentration - 3x

| Variable | P-value | Statistical Support |
|---|---|---|
| (Supplement @ 3x) | (lower the better) | (Hypothesis 2) |
| Ginkgo biloba | 3.68328E−06 | Strong Support |
| | | (supplement is beneficial) |
| Resveratrol | 1.34618E−04 | Strong Support |
| | | (supplement is beneficial) |
| Glutathione | 0.335751056 | No Support |
| | | (may or may not be beneficial) |

Western Blot Test—Hypothesis 3a: Strongly Supported for Ginkgo biloba

The Western Blot Test for GCLc quantification supported the hypothesis that dietary supplements can induce over expression of the GCLc gene, increasing the levels of the GCLc enzyme. The highest effect was noticed at three times (3×) concentration, with decreased GCLc levels at six times dosage implying toxicity at higher levels as seen in FIG. 4.
1. Ginkgo biloba—upto 14% increase in the GCLc enzyme at the optimal dose, compared to the control, indicating that epigenetic over expression of the GCLc genes occur causing increased enzyme levels.
2. Glutathione—7% increase in GCLc.
3. Resveratrol—Only 1% increase in GCLc, showing no effect on the GCLc gene, implies other genetic pathways of increasing longevity.
   The Glutathione Fluorimetric Assays: Hypothesis 3b—Strongly Supported

The fluorimetric assays shown in FIG. 5 strongly supported the hypothesis that dietary supplements can increase glutathione synthesis. Three times dosage was optimal in all supplements and six times dosage was toxic with significant negative feedback.
1. Ginkgo biloba—Increased Glutathione by up to 122% showing glutathione synthesis through GCLc pathways.
2. Glutathione—154% increase in glutathione. In spite of this, the effects on life span are not as impressive, probably due to inadequate Glutathione absorption. The test may not be accurate as the test may be quantifying the exogenous glutathione on the fly bodies from the diet.
3. Resveratrol—Only 1% increase in GCLc but increase in Glutathione by 100% suggesting Glutathione synthesis through other pathways such as Sirtuin.

Together, these results suggest that the increase in longevity induced by supplements, such as Ginkgo biloba is directly from the over expression of the GCLc gene, and the resultant de novo Glutathione synthesis. The increase in lifespan by Resveratrol is also through Glutathione synthesis but through other enzymatic pathways as the GCLc levels were unchanged. A diet that includes these antioxidants can increases resistance to oxidative damage and help prolong healthy years of life.

In another aspect of the present invention, the amyloid, oxidative stress and mitochondrial cascade theories were integrated to research the cellular mechanisms underlying neuro-degenerative diseases. A transgenic Drosophila with pan-neuronal over-expression of GCLc gene in Aβ42 model Alzheimer flies was created. The Aβ42-GCLc Drosophila were subjected to redox stress through sleep deprivation and compared with Aβ42 Drosophila. Natural yellow-white Drosophila was used as positive control. The mitochondrial enzymatic activities of the Electron Transport Chains (ETC) of the Drosophila groups were analyzed using colorimetric enzymatic assays.

The results showed that the lifespan of Aβ42-GCLc Drosophila were significantly higher than those of Aβ42 flies, especially under redox stress. The ETC assays indicated that Aβ42-GCLc had improved mitochondrial enzyme activity in all enzymatic complexes, especially in I, III and IV complexes. These complexes are encoded by mtDNA. These results suggest an increased in vivo Glutathione synthesis in the Aβ42-GCLc Drosophila, and enhanced protection of mitochondrial DNA from neurotoxicity. Therefore, it may be concluded that antioxidant genes such as GCLc may play an important role in neuroprotection under conditions of oxidative stress, aging and neurodegerative disorders.

In an embodiment of the present invention, natural yellow-white Drosophila melanogaster, transgenic GAL4-GCLc flies, and Alzheimer model Aβ42 (elav-GAL4; UAS Aβ42/cyo) Drosophila melanogaster flies were obtained from different sources and bred in the laboratory.

The Aβ42-GCLc Drosophila was prepared by cross-breeding virgin female Alzheimer elav-Aβ42 flies with male GAL4-GCLc flies. Multiple culture bottles were maintained till a large sample subject population was obtained. It was observed that the cross bred Aβ42-GCLc Drosophila were stable and showed traits of both Aβ42 and GCLc across generations.

In various aspects of the present invention, the following hypotheses were proposed and tested using scientific methods:

### Hypothesis 1: Genetic Modulation

If lifespans of Alzheimer's model Aβ42 Drosophila and Aβ42-GCLc are studied, the Aβ42-GCLc group will outlive the Aβ42 group of flies; all groups will exhibit shorter lifespans compared to the control group of yellow-white Drosophila melanogaster.

### Hypothesis 2: Resistance to Redox Stress

If Aβ42 and Aβ42-GCLc Drosophila are subjected to continuous oxidative stress, the Aβ42-GCLc flies will be able to better resist stress, as exhibited by longer lifespan; all groups will exhibit shorter lifespans compared to the control group of yellow-white Drosophila melanogaster.

### Hypothesis 3: Mitochondrial Bioenergetics

If the Mitochondrial Electron Transport Chain (ETC) of Aβ42 and Aβ42-GCLc groups of Drosophila is analyzed, Aβ42-GCLc will exhibit more enzymatic activity in the mitochondrial complexes compared to Aβ42; all groups will exhibit lower enzyme activity compared to the control group of yellow-white Drosophila melanogaster.

In an embodiment of the present invention, male and female yellow-white Drosophila, Alzheimer model “c155-elav/elav GAL4; UAS-Aβ42/cyo” Drosophila and GCLc-GAL4 Drosophila melanogaster parent flies were obtained. A total of about 1000 subject Drosophila were used for this study.

### Animal Model Preparation

The flies were bred in Drosophila glass culture bottles with Carolina blue medium 424 formula, activated yeast and water. The Alzheimer's/GCLc transgenic fly crosses were prepared by breeding virgin female Alzheimer's elav-GAL4; UAS-Aβ42 flies with male GCLc-GAL4 flies. Once the larvae hatched, they were immediately isolated, anesthetized by a CO₂ anesthetizer, and divided on an ice pack by gender. Only male flies were used as the subject population.

The subject flies were divided into 100 flies each in four vials of about 25 each. There were 3 types of flies that included male yellow white, male Aβ42 Alzheimer's and male Aβ42/GCLc. Three study groups in each type of fly were formed—one group was used for natural lifespan, one for redox stress lifespan and one for the mitochondrial ETC assays.

### Redox Stress Tests:

The 24 hour redox stress tests were carried out by constant oscillation, noise and light in each group. The non-stressed flies were in an insect culture incubator with no movement, natural noise and light.

### Life Span Tests:

The 40 vials of 25 flies each were changed every other day. The right food for every vial (Carolina 424 blue formula with dry activated yeast and water) was made and the flies from one vial to the other were transferred. The number of flies that died each time was recorded. This was continued until the last fly died in each vial. The average 50% survival life spans for each group of flies in each group (in each of the 4 vials) were recorded, tabulated, graphed and tested for statistical significance using the two tailed T test, analyzed and conclusions were drawn.

Further, the ETC subject group of flies were collected and frozen, 15 each time, at four weeks for further testing. The Mitochondrial Electron Transport Chain (ETC) colorimetric enzymatic assays and analysis was performed.

### Homogenizing:

For each group of Drosophila studied i.e., yellow white, Aβ42, and Aβ42-GCLc, 15 flies from each group were frozen at four weeks of their lifespan. The flies were taken on dry ice for the ETC enzymatic assays using the spectrophotometer. The cultured cell sonification medium was localized and a pipette was used to measure 300 microliters of solution for each Eppendorf tube containing flies. The solution and flies were combined in the pre-cleaned tubes and homogenized with a pipette until liquefied, then returned into the eppendorf tubes until each study group was done. The eppendorf tubes were spun at 2500 rpm in a cold centrifuge for about 20 minutes and the lysate was transferred.

### Standardizing:

A solution including 2 microliters of BSA or protein of 10 mg per mL with 18 microliters of the buffer was prepared. Each solution made from the reagents and lysates for the respective complexes in the 96-well plate was mixed and connected to the computer for data recording and analysis.

### Set-Up:

The activities of complex II (succinate dehydrogenase), total and rotenone sensitive complex I+III (NADH: Cytochrome C Oxido reductase), and complex IV (cytochrome C oxidase), and CS (Citrate Synthase) were measured using appropriate electron acceptors/donors. Each assay was performed in duplicate. A skilled artisan will appreciate that the procedure for such enzymatic assays are well known in the art, and their description has been omitted so as not to obfuscate the present specification.

### Colorimetric Assays:

ETC enzymes were assayed at 30° C. using a temperature-controlled spectrophotometer; Ultraspec 6300 PRO, from Biochrom Ltd., (Cambridge, UK). The increase or decrease in the absorbance of cytochrome C at 550 nm was measured for complex I+III, II+III, and complex IV. For complex II, the reduction of 2, 6-dichloroindophenol (DCIP) at 600 nm was measured. Citrate Synthase (CS) was used as a marker for mitochondrial content and was measured by reduction of Ellman's reagent at 412 nm. Enzyme activities were expressed as nmol/min/mg protein. Each of the data was analyzed for complexes I+III, II, II+III, IV, and Citrate Synthase. The spectrophotometer readings in nmol/min/mg were adjusted for differences in Citrate Synthase and shown as percentage of readings for yellow-white control. It will be appreciated by a person skilled in the art that such calorimetric assays are also well known in the art and numerous other modifications in the procedures may be made without departing from scope and spirit of the present invention.

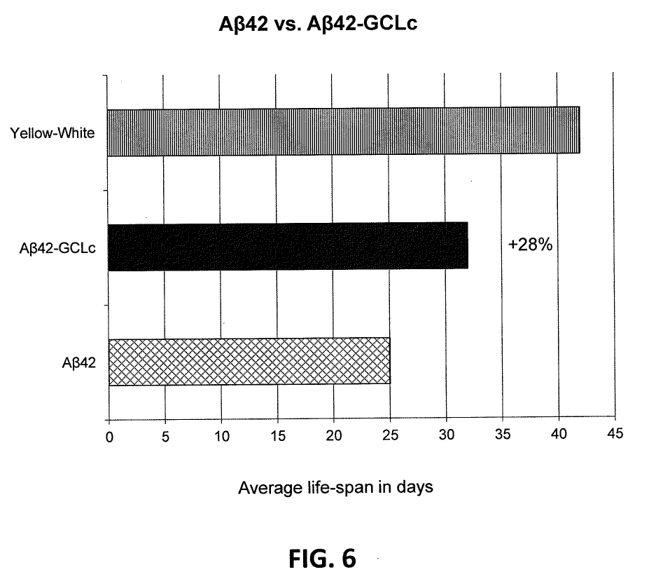

Referring now FIG. 6, a graph illustrating average life-span in days for yellow-white, Aβ42, and Aβ42-GCLc Drosophila is shown, in accordance with an exemplary test. The lifespans of 200 male flies each of yellow-white, Aβ42, and Aβ42-GCLc Drosophila melanogaster were studied and the average lifespan was taken to plot the graph. It can be observed from the graph that the GCLc overexpression caused an increase in the lifespan of about 28% as compared to that of the Aβ42 flies.

Thus, the Hypothesis 1 (of Genetic Modulation) is strongly supported. The Alzheimer flies had a 40% decrease in lifespan compared to the yellow whites. However, the increase in the Aβ42-GCLc group was found significant at 99% confidence level.

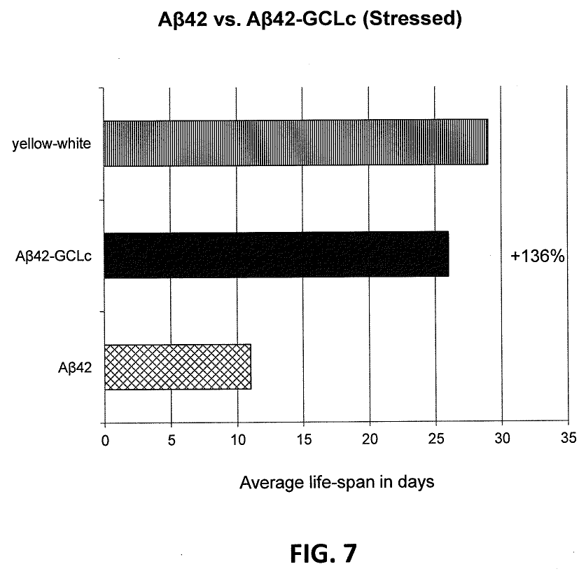

FIG. 7 is a graph illustrating average life-span in days for stressed yellow-white, Aβ42, and Aβ42-GCLc Drosophila, in accordance with an exemplary test. As described in the foregoing specifications, 24 hour redox stress tests were carried out by constant oscillation, noise and light in each group. It can be seen from the graph that the stress was extremely significant in the Aβ42 Drosophila. The stress decreased the average lifespan of the Aβ42 Drosophila to 11 days. However, the average lifespan of the Aβ42-GCLc Drosophila was reduced only to 26 days, i.e., a 136% increase as compared to that of the Aβ42 Drosophila. This observation confirmed that increased Glutathione levels can mitigate the redox stress, especially in Alzheimer's flies.

Thus, the hypothesis 2 (of resistance to redox stress) was strongly supported. The Alzheimer's flies had a 60% decrease in lifespan when stressed. However, the Aβ42-GCLc group under stress had an increase of 136% in lifespan compared to the stressed Alzheimer's group. The increased average lifespan was found significant at the 99% confidence level.

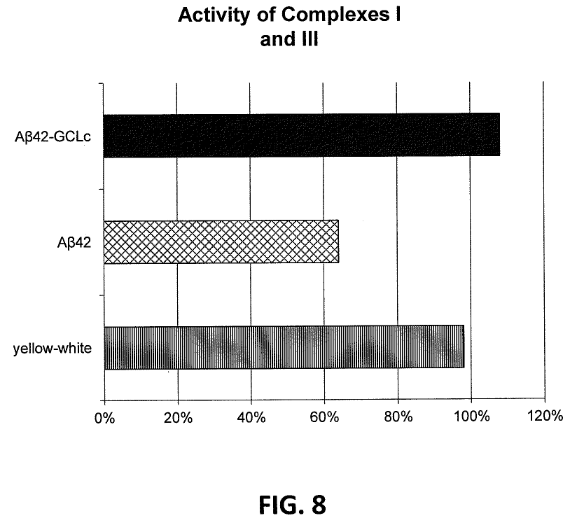

FIG. 8 is a graph illustrating the activity of complexes I and III for yellow-white, Aβ42, and Aβ42-GCLc Drosophila, in accordance with an exemplary ETC assay. As described in the foregoing, an enzymatic assay was conducted to observe the activities of NADH dehydrogenase and cytochrome C oxidase reductase (mitochondrial complexes I+III). Aβ42-GCLc Drosophila exhibited a higher complex I+III activity as compared to that of the normal yellow-white Drosophila melanogaster.

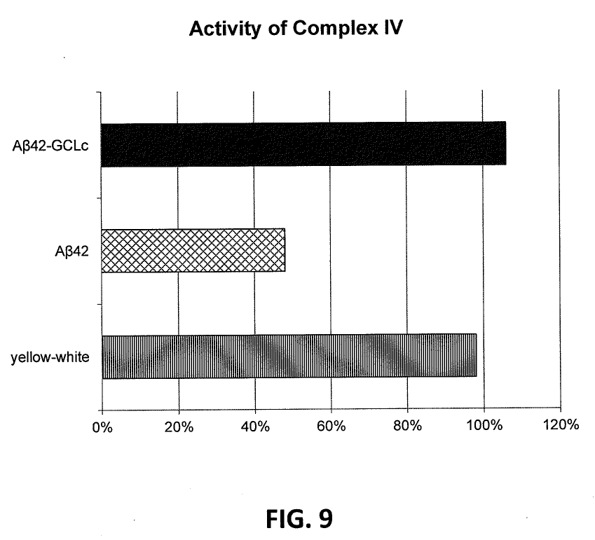

FIG. 9 is a graph illustrating the activity of complex IV for yellow-white, Aβ42, and Aβ42-GCLc Drosophila, in accordance with an exemplary ETC assay. As described in the foregoing, an enzymatic assay was conducted to observe the activity of cytochrome C oxidase (mitochondrial complex IV). As hypothesized, Aβ42-GCLc flies exhibited dramatically more activity in mitochondrial complex IV compared to that in Aβ42 flies. The GCLc activity in the Alzheimer flies restored and even increased complex IV activity compared to the control group of yellow-white Drosophila melanogaster.

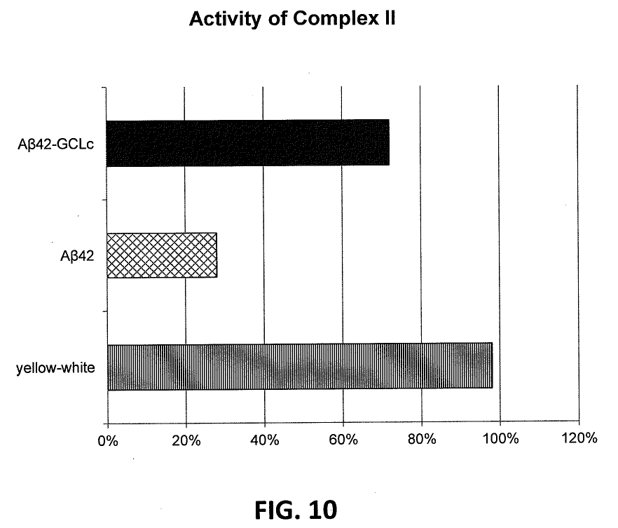

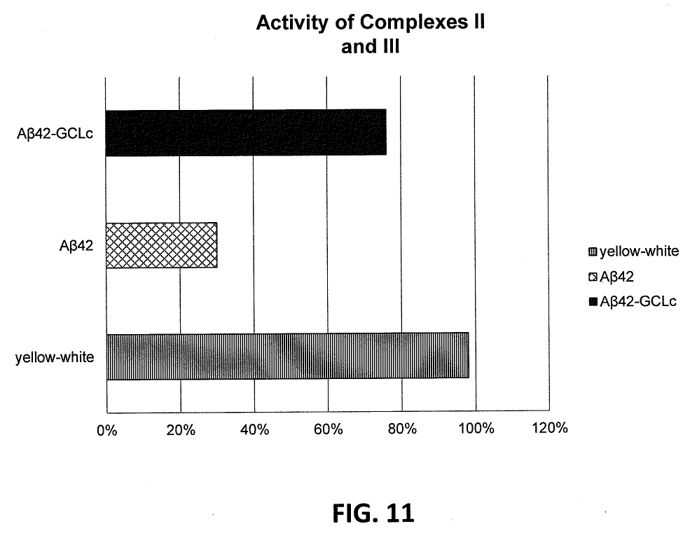

FIG. 10 is a graph illustrating the activity of complex II for yellow-white, Aβ42, and Aβ42-GCLc Drosophila, in accordance with an exemplary ETC assay. As described in the foregoing, an enzymatic assay was conducted to observe the activity of succinate dehydrogenase (mitochondrial complex II). FIG. 6 is a graph illustrating the activity of complexes II and III for yellow-white, Aβ42, and Aβ42-GCLc Drosophila, in accordance with an exemplary ETC assay conducted to observe the activities of succinate dehydrogenase and cytochrome C oxidase reductase (mitochondrial complexes II+III). As can be seen from FIG. 10 and FIG. 11, the enzymatic activities of complexes II and II+III in the Aβ42-GCLc flies were significantly higher than those in the Aβ42 flies.

The Mitochondrial activities in all four complexes tested were significantly lower in the Alzheimer flies compared to the yellow-whites, reduced by 40-70%. However, in each complex tested, the Aβ42-GCLc flies' enzyme activity was more than doubled as compared to the Alzheimer's flies. In complex I+III and IV, the enzymatic activities in Aβ42-GCLc flies were even higher than the yellow-white flies, suggesting increased protection for mtDNA. Thus, hypothesis 3 (of mitochondrial bioenergetics) was supported by these observations.

Various embodiments of the present invention confirm the importance of redox stress on health and longevity and that dietary supplementation to induce epigenetic changes is a part of the solution. Antioxidant genes, such as GCLc, may be able to mitigate some of the negative effects of stress on health and longevity, especially in people with neurodegenerative diseases of aging, such as AD. Patients with Alzheimer's with decreased glutathione levels may have a decreased innate capacity to withstand stress. Over-expressing the GCLc genes through epigenetic intervention may be a potential biotechnology application for the future. Serum glutathione levels in patients, who suffer from stress, sleep deprivation and Alzheimer's can be tested. Humans require 250 milligrams of Glutathione a day; the average American consumes less than 35 mg. Over the counter, Glutathione supplements could substitute the 60 million prescriptions every year for sleep disorders and may be a valuable supplement in delaying AD. As glutathione is not absorbed well into the body, glutathione precursors such as N-acetyl-cysteine (NAC), Selenium and Vitamin C or the in vivo Glutathione inducing dietary supplements such as Ginkgo biloba, Resveratrol, NAC etc. are good alternatives. Many of these supplements have been marketed by biotechnology and pharmaceutical companies, further experimentation may lead to marketing and commercialization of these supplements may be taken. Glutathione-rich foods such as asparagus, mangoes, eggs, garlic and whey protein would also help. Increasing Glutathione (GSH) levels in the brain by exploring new molecular targets and transcription factors may be a focus area for such research.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A living organism that expresses enhanced levels of an enzyme associated with antioxidant synthesis, wherein the living organism displays at least one phenotype associated with a neurodegenerative disorder.

2. The living organism of claim 1, wherein the enhanced level of the enzyme is induced by using at least one dietary supplement.

3. The living organism of claim 2, wherein the at least one dietary supplement is selected from the group comprising: Ginkgo biloba, Resveratrol, Glutathione, Superoxide dismutase, Coenzyme Q10, N-Acetyl Cysteine, Whey Protein, Ginger, Garlic, Green Tea and Turmeric.

4. The living organism of claim 2, wherein the at least one dietary supplement is selected from the group comprising: Ginkgo biloba, Resveratrol, and Glutathione.

5. The living organism of claim 1, wherein the at least one phenotype is caused due to at least one of an injection of beta-amyloid 42 in the living organism, an overexpression of beta-amyloid 42 in the living organism, and by virtue of the neurodegenerative disorder.

6. The living organism of claim 1 is a transgenic organism.

7. The living organism of claim 1 is a transgenic fly.

8. The living organism of claim 1 is a transgenic fly of Drosophila genus.

9. The living organism of claim 1, wherein the antioxidant is glutathione (GSH).

10. The living organism of claim 1, wherein the enzyme is glutamate-cysteine ligase (GCL).

11. The living organism of claim 1, wherein the neurodegenerative disorder is Alzheimer's disease.

12. A method for diagnosing a neurodegenerative disorder in a first living organism, the method comprising:
assessing an enzymatic activity of at least one mitochondrial enzyme in a first sample isolated from the first living organism; and
comparing the enzymatic activity of the at least one mitochondrial enzyme with an enzymatic activity of the at least one mitochondrial enzyme in a second sample isolated from a second living organism known to be free of the neurodegenerative disorder,
wherein a difference between the first enzymatic activity and the second enzymatic activity indicates a presence of the neurodegenerative disorder in the first living organism.

13. The method of claim 9, wherein the neurodenerative disorder is Alzheimer's disease.

14. The method of claim 9, wherein the at least one mitochondrial enzyme is a mitochondrial complex enzyme selected from a group consisting of NADH dehydrogenase, succinate dehydrogenase, cytochrome C oxido reductase, cytochrome C oxidase.

15. A method for at least one of preventing and treating a neurodegenerative disorder in a living organism, the method comprising:
increasing a level of glutathione in the living organism.

16. The method of claim 15, wherein the level of glutathione in the living organism is increased by modulating an expression of an enzyme associated with glutathione synthesis.

17. The method of claim 15, wherein the enzyme associated with glutathione synthesis is glutamate-cysteine ligase (GCL).

18. The method of claim 15, wherein the neurodegenerative disorder is Alzheimer's disease.

19. The method of claim 15, wherein the increased level of glutathione is induced by using at least one dietary supplement.

20. The method of claim 15, wherein the at least one dietary supplement is selected from a group comprising Ginkgo biloba, Resveratrol, Glutathione, Superoxide dismutase, Coenzyme Q10, N-Acetyl Cysteine, Whey Protein, Ginger, Garlic, Green Tea, and Turmeric.
